(19) United States
(12) Patent Application Publication
(10) Pub. No.: US 2018/0131218 A1
(43) Pub. Date: May 10, 2018

(54) WIRLESS POWER TRANSFER FOR A WIRELESS POWER RECEIVER WITH A DEAD BATTERY

(71) Applicant: QUALCOMM Incorporated, San Diego, CA (US)

(72) Inventors: Stephen Jay SHELLHAMMER, Ramona, CA (US); William Henry VON NOVAK, III, San Diego, CA (US); Mark WHITE, II, Solana Beach, CA (US)

(21) Appl. No.: 15/348,049

(22) Filed: Nov. 10, 2016

(51) Int. Cl. H02J 7/02; A61N 1/378

(52) U.S. Cl. CPC H02J 7/025; H02J 2007/0096; A61N 1/3787

(57) ABSTRACT

Certain aspects of the present disclosure generally relate to methods and apparatus for wirelessly charging a device having a wireless power receiver with a dead battery. One example method for safely wirelessly charging an implantable device, with an apparatus, generally includes determining that the implantable device has a dead battery; based on the determination, wirelessly transmitting power from the apparatus at an initial level for a first interval; checking for a first signal received from the implantable device during or at an end of the first interval or a period associated with the initial level; and if no first signal is received from the implantable device, increasing the transmitted power to a higher level for a second interval.

### TECHNICAL FIELD

The present disclosure generally relates to wireless power transfer and, more specifically, to wirelessly charging a wireless power receiver with a dead battery.

### BACKGROUND

An increasing number and variety of electronic devices are powered via rechargeable batteries. Such devices include mobile phones, portable music players, laptop computers, tablet computers, computer peripheral devices, communication devices (e.g., Bluetooth devices), digital cameras, hearing aids, medical implants, and the like. While battery technology has improved, battery-powered electronic devices increasingly demand and consume greater amounts of power. As such, these devices constantly require recharging. Rechargeable devices are often charged via wired connections that employ cables or other similar connectors that are physically connected to a power supply. Cables and similar connectors may sometimes be inconvenient or cumbersome and have other drawbacks. Wireless power transfer systems, for example, may allow users to charge and/or power electronic devices without physical, electrical connections, thus reducing the number of components involved for operation of the electronic devices and simplifying the use thereof.

For example, some battery-powered devices, such as medical implants (e.g., pacemakers, neuromodulation devices, insulin pumps, etc.) may be located in areas where replacing the battery is not always feasible (e.g., in a body, such as a human body). For example, to change a battery for a medical implant, surgery may need to be performed, which is risky. Accordingly, it may be safer to charge such devices wirelessly.

Further, some electronic devices may not be battery powered, but it still may be beneficial to utilize wireless power transfer to power such devices. In particular, the use of wireless power may eliminate the need for cords or cables to be attached to the electronic devices, which may be inconvenient and aesthetically displeasing.

Different electronic devices may have different shapes, sizes, and power specifications. There is flexibility in having different sizes and shapes in the components (e.g., magnetic coil, charging plate, etc.) that make up a wireless power transmitter and/or a wireless power receiver in terms of industrial design and support for a wide range of devices.

### SUMMARY

Certain aspects of the present disclosure provide a method for safely wirelessly charging an implantable device, with an apparatus. The method generally includes determining that the implantable device has a dead battery; based on the determination, wirelessly transmitting power from the apparatus at an initial level for a first interval; checking for a first signal received from the implantable device during or at an end of the first interval or a period associated with the initial level; and if no first signal is received from the implantable device, increasing the transmitted power to a higher level for a second interval.

Certain aspects of the present disclosure provide an apparatus for safely wirelessly charging an implantable device. The apparatus generally includes a power transmitting element for wirelessly transmitting power, transmit circuitry coupled to and configured to drive the power transmitting element, and a processing system coupled to the transmit circuitry. The processing system is configured to determine that the implantable device has a dead battery; to control the transmit circuitry to wirelessly transmit power from the power transmitting element at an initial level for a first interval, based on the determination of the dead battery; to check for a first signal received from the implantable device during or at an end of the first interval or a period associated with the initial level; and to control the transmit circuitry to increase the wirelessly transmitted power to a higher level for a second interval, if no first signal is received from the implantable device.

Certain aspects of the present disclosure provide a non-transitory computer-readable medium for safely wirelessly charging an implantable device, with an apparatus. The computer-readable medium generally includes instructions executable by a processing system to determine that the implantable device has a dead battery; to control wirelessly transmitting power from the apparatus at an initial level for a first interval, based on the determination; to check for a first signal received from the implantable device during or at an end of the first interval or a period associated with the initial level; and to control increasing the wirelessly transmitted power to a higher level for a second interval, if no first signal is received from the implantable device.

Certain aspects of the present disclosure provide a first apparatus for safely wirelessly charging a second apparatus. The first apparatus generally includes means for determining that the second apparatus has a dead battery; means for wirelessly transmitting power from the first apparatus to the second apparatus, the means for wirelessly transmitting power being configured to wirelessly transmit power at an initial level for a first interval, based on the determination of the dead battery; and means for checking for a first signal received from the second apparatus during or at an end of the first interval or a period associated with the initial level. The means for wirelessly transmitting power is further configured to increase the transmitted power to a higher level for a second interval, if no first signal is received from the second apparatus.

Certain aspects of the present disclosure provide a method for wirelessly charging a wireless power receiver, with a wireless power transmitter. The method generally includes determining that the wireless power receiver has a dead battery; based on the determination, wirelessly transmitting power from the wireless power transmitter to the wireless power receiver at an initial level; gradually increasing the wirelessly transmitted power from the initial level until an overvoltage protection (OVP) condition is detected from the wireless power receiver; based on the detection of the OVP condition, reducing the wirelessly transmitted power from a charging level associated with the detection of the OVP condition to a backoff level; and wirelessly charging the wireless power receiver at the backoff level.

Certain aspects of the present disclosure provide a wireless power transmitter for wirelessly charging a wireless power receiver. The wireless power transmitter generally includes a power transmitting element for wirelessly transmitting power, transmit circuitry coupled to and configured to drive the power transmitting element, and a processing system coupled to the transmit circuitry. The processing system is generally configured to determine that the wireless power receiver has a dead battery; to control the transmit circuitry to wirelessly transmit power from the power transmitting element at an initial level, based on the determination of the dead battery; to control the transmit circuitry to gradually increase the wirelessly transmitted power from the initial level until an OVP condition is detected from the wireless power receiver; to control the transmit circuitry to reduce the wirelessly transmitted power from a charging level associated with the detection of the OVP condition to a backoff level, based on the detection of the OVP condition; and to control the transmit circuitry to wirelessly charge the wireless power receiver at the backoff level.

Certain aspects of the present disclosure provide a non-transitory computer-readable medium for wirelessly charging a wireless power receiver, with a wireless power transmitter. The computer-readable medium generally includes instructions executable by a processing system to determine that the wireless power receiver has a dead battery; to wirelessly transmit power from the wireless power transmitter to the wireless power receiver at an initial level, based on the determination; to gradually increase the wirelessly transmitted power from the initial level until an OVP condition is detected from the wireless power receiver; to reduce the wirelessly transmitted power from a charging level associated with the detection of the OVP condition to a backoff level, based on the detection of the OVP condition; and to wirelessly charge the wireless power receiver at the backoff level.

Certain aspects of the present disclosure provide a first apparatus for wirelessly charging a second apparatus. The first apparatus generally includes means for determining that the second apparatus has a dead battery, means for wirelessly transmitting power to the second apparatus, and means for detecting an OVP condition from the second apparatus. The means for wirelessly transmitting power is configured to wireless transmit power at an initial level based on the determination of the dead battery; to gradually increase the wirelessly transmitted power from the initial level until the OVP condition is detected; to reduce, based on the detection of the OVP condition, the wirelessly transmitted power from a charging level associated with the detection of the OVP condition to a backoff level; and to wirelessly charge the second apparatus at the backoff level.

Certain aspects of the present disclosure provide a method for wirelessly charging a wireless power receiver, with a wireless power transmitter. The method generally includes determining a default level for wirelessly charging the wireless power receiver based on data characterizing wireless charging of the wireless power receiver while a battery of the wireless power receiver is charging well; determining that the battery of the wireless power receiver is dead; and based on the determination of the dead battery, wirelessly transmitting power from the wireless power transmitter at the default level.

Certain aspects of the present disclosure provide a wireless power transmitter for wirelessly charging a wireless power receiver. The wireless power transmitter generally includes a power transmitting element for wirelessly transmitting power, transmit circuitry coupled to and configured to drive the power transmitting element, and a processing system coupled to the transmit circuitry. The processing system is generally configured to determine a default level for wirelessly charging the wireless power receiver based on data characterizing wireless charging of the wireless power receiver while a battery of the wireless power receiver is charging well; to determine that the battery of the wireless power receiver is dead; and to control the transmit circuitry to wirelessly transmit power from the power transmitting element at the default level, based on the determination of the dead battery.

Certain aspects of the present disclosure provide a non-transitory computer-readable medium for wirelessly charging a wireless power receiver, with a wireless power transmitter. The computer-readable medium generally includes instructions executable by a processing system to determine a default level for wirelessly charging the wireless power receiver based on data characterizing wireless charging of the wireless power receiver while a battery of the wireless power receiver is charging well; to determine that the battery of the wireless power receiver is dead; and to control wireless transmission of power from the wireless power transmitter at the default level, based on the determination of the dead battery.

Certain aspects of the present disclosure provide a first apparatus for wirelessly charging a second apparatus. The first apparatus generally includes means for determining a default level for wirelessly charging the second apparatus based on data characterizing wireless charging of the second apparatus while a battery of the second apparatus is charging well; means for determining that the battery of the second apparatus is dead; and means for wirelessly transmitting power from the first apparatus to the second apparatus at the default level, based on the determination of the dead battery.

The following detailed description and accompanying drawings provide a better understanding of the nature and advantages of the present disclosure.

### BRIEF DESCRIPTION OF THE DRAWINGS

With respect to the discussion to follow and in particular to the drawings, it is stressed that the particulars shown represent examples for purposes of illustrative discussion, and are presented in the cause of providing a description of principles and conceptual aspects of the present disclosure. In this regard, no attempt is made to show implementation details beyond what is needed for a fundamental understanding of the present disclosure. The discussion to follow, in conjunction with the drawings, makes apparent to those of skill in the art how aspects in accordance with the present disclosure may be practiced. In the accompanying drawings:

FIG. 1 is a functional block diagram of an example wireless power transfer system, in accordance with certain aspects of the present disclosure.

FIG. 2 is a more-detailed block diagram of an example wireless power transfer system, in accordance with certain aspects of the present disclosure.

FIG. 3 is a schematic diagram of a portion of example transmit or receive circuitry of FIG. 2 including a power transmitting or receiving element, in accordance with certain aspects of the present disclosure.

FIGS. 4A and 4B conceptually illustrate wirelessly charging an implantable device at two different depths in a body, in accordance with certain aspects of the present disclosure.

FIGS. 5A and 5B conceptually illustrate wirelessly charging an implantable device with two different orientations in a body, in accordance with certain aspects of the present disclosure.

FIG. 6 is a flowchart of example operations for safely wirelessly charging an implantable device when no signal from the device is detected, in accordance with certain aspects of the present disclosure.

FIG. 7 conceptually illustrates concurrently wirelessly charging multiple implantable devices in the same body, in accordance with certain aspects of the present disclosure.

FIG. 8 is a flow diagram of example operations for safely wirelessly charging an implantable device, in accordance with certain aspects of the present disclosure.

FIG. 9 is a flowchart of example operations for wirelessly charging a wireless power receiver according to stored data, in accordance with certain aspects of the present disclosure.

FIG. 10 is a flow diagram of example operations for wirelessly charging a wireless power receiver according to a default level, in accordance with certain aspects of the present disclosure.

FIG. 11 is a flowchart of example operations for ramping up power until overvoltage protection (OVP) is detected and then backing off, in accordance with certain aspects of the present disclosure.

FIG. 12 is a flow diagram of example operations for wirelessly charging a wireless power receiver according to a backoff level, in accordance with certain aspects of the present disclosure.

### DETAILED DESCRIPTION

Drawing elements that are common among the following figures may be identified using the same reference numerals.

Wireless power transfer may refer to transferring any form of energy associated with electric fields, magnetic fields, electromagnetic fields, or otherwise from a transmitter to a receiver without the use of physical electrical conductors (e.g., power may be transferred through free space or air). The power output into a wireless field (e.g., a magnetic field or an electromagnetic field) may be received, captured by, or coupled by a “power receiving element” to achieve power transfer.

### Example Wireless Power Transfer System

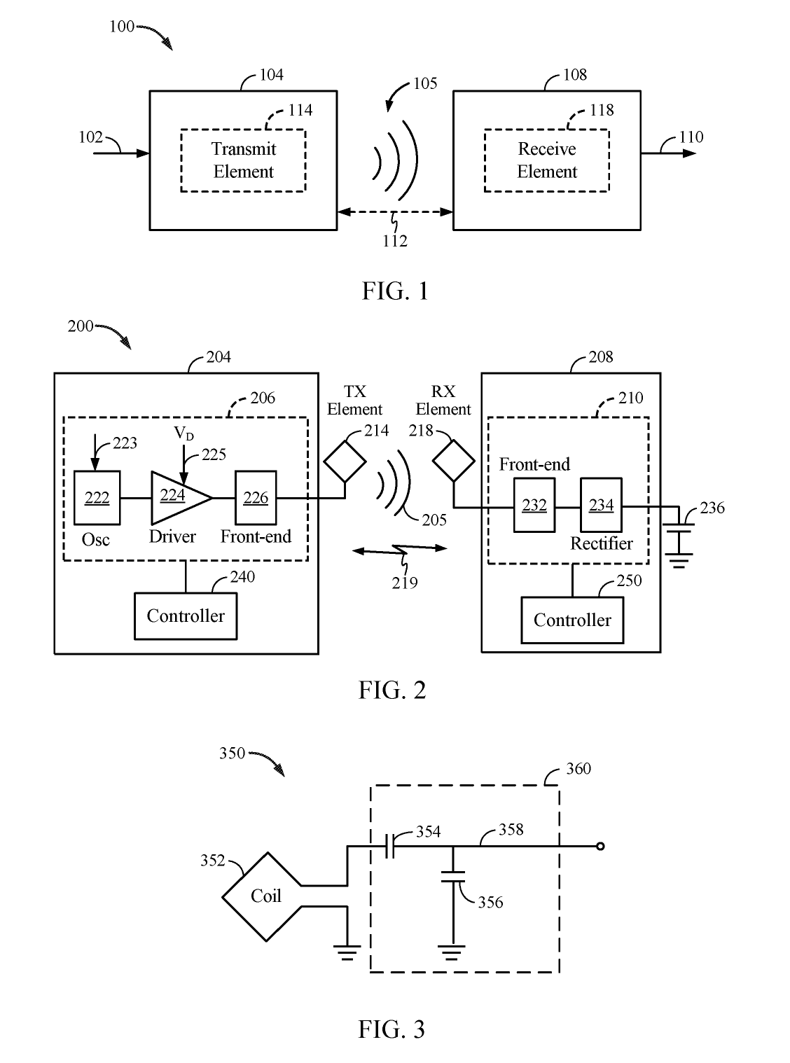

FIG. 1 is a functional block diagram of an example wireless power transfer system 100, in accordance with certain aspects of the present disclosure. Input power 102 may be provided to a transmitter 104 from a power source (not shown in this figure) to generate a wireless (e.g., magnetic or electromagnetic) field 105 for performing energy transfer. A receiver 108 may be subjected to the wireless field 105 and generate output power 110 for storing or consumption by a device (not shown in this figure) coupled to the output power 110. The transmitter 104 and the receiver 108 may be separated by a distance 112. The transmitter 104 may include a power transmitting element 114 for transmitting/providing energy to the receiver 108. The receiver 108 may include a power receiving element 118 for receiving/capturing energy transmitted from the transmitter 104.

In one illustrative aspect, the transmitter 104 and the receiver 108 may be configured according to a mutual resonant relationship. When the resonant frequency of the receiver 108 and the resonant frequency of the transmitter 104 are substantially the same or very close, transmission losses between the transmitter 104 and the receiver 108 are reduced. As such, wireless power transfer may be provided over larger distances. Resonant inductive coupling techniques may thus allow for increased efficiency and power transfer over various distances and with a variety of inductive power transmitting and receiving element configurations.

In certain aspects, the wireless field 105 may correspond to the “near field” of the transmitter 104. The near field may correspond to a region in which there are strong reactive fields resulting from the currents and charges in the power transmitting element 114 that minimally radiate power away from the power transmitting element 114. The near field may correspond to a region that is within about one wavelength (or a fraction thereof) of the power transmitting element 114. Conversely, the far field may correspond to a region that is greater than about one wavelength of the power transmitting element 114.

In certain aspects, efficient energy transfer may occur by coupling a large portion of the energy in the wireless field 105 to the power receiving element 118, rather than propagating most of the energy in an electromagnetic wave to the far field.

In certain implementations, the transmitter 104 may output a time-varying magnetic (or electromagnetic) field with a frequency corresponding to the resonant frequency of the power transmitting element 114. When the receiver 108 is within the wireless field 105, the time-varying magnetic (or electromagnetic) field may induce a current in the power receiving element 118. As described above, if the power receiving element 118 is configured as a resonant circuit to resonate at (or very close to) the frequency of the power transmitting element 114, energy may be efficiently transferred. An alternating current (AC) signal induced in the power receiving element 118 may be rectified to produce a direct current (DC) signal that may be provided to charge or to power a load.

FIG. 2 is a more-detailed block diagram of an example wireless power transfer system 200, in accordance with certain aspects of the present disclosure. The system 200 may include a transmitter 204 and a receiver 208. The transmitter 204 (also referred to herein as a power transfer unit, or PTU) may include transmit circuitry 206 that may include an oscillator 222, a driver circuit 224, and a front-end circuit 226. The oscillator 222 may be configured to generate an oscillator signal (also known as an oscillating signal) at a desired frequency (e.g., fundamental frequency), which may be adjusted in response to a frequency control signal 223. The oscillator 222 may provide the oscillator signal to the driver circuit 224. The driver circuit 224 may be configured to drive the power transmitting element 214 at, for example, a resonant frequency of the power transmitting element 214, according to the frequency of the oscillator signal. The power transmitting element 214 may be powered by a power supply signal (V_{D}) 225. The driver circuit 224 may be a switching amplifier configured to receive a square wave from the oscillator 222 and output a sine wave as a driving signal output.

The front-end circuit 226 may include a filter circuit configured to filter out harmonics or other unwanted frequencies. The front-end circuit 226 may also include a matching circuit configured to match the impedance of the transmitter 204 to the impedance of the power transmitting element 214 in an effort to reduce power loss. As will be explained in more detail below, the front-end circuit 226 may include a tuning circuit to create a resonant circuit with the power transmitting element 214. As a result of driving the power transmitting element 214, the power transmitting element 214 may generate a wireless field 205 to wirelessly output power at a level sufficient for charging a battery 236, or otherwise powering a load.

The transmitter 204 may further include a controller 240 operably coupled to the transmit circuitry 206 and configured to control one or more aspects of the transmit circuitry 206, or accomplish other operations relevant to managing the transfer of power. The controller 240 may be a microcontroller or a processor, for example. For certain aspects, the controller 240 may be implemented as an application-specific integrated circuit (ASIC). The controller 240 may be operably connected, directly or indirectly, to each component of the transmit circuitry 206. The controller 240 may be further configured to receive information from each of the components of the transmit circuitry 206 and perform calculations based on the received information. The controller 240 may be configured to generate control signals (e.g., signal 223) for each of the components that may adjust the operation of that component. As such, the controller 240 may be configured to adjust or manage the power transfer based on a result of the operations performed by it. The transmitter 204 may further include a memory (not shown) configured to store data, such as instructions for causing the controller 240 to perform particular functions, such as those related to management of wireless power transfer.

The receiver 208 (also referred to herein as a power receiving unit, or PRU) may include receive circuitry 210 that may include a front-end circuit 232 and a rectifier circuit 234. The front-end circuit 232 may include matching circuitry configured to match the impedance of the receive circuitry 210 to the impedance of the power receiving element 218 in an effort to reduce power loss. As will be explained below, the front-end circuit 232 may further include a tuning circuit to create a resonant circuit with the power receiving element 218. The rectifier circuit 234 may generate a DC power output from an AC power input to charge the battery 236, as shown in FIG. 2, or power a load. The receiver 208 and the transmitter 204 may additionally communicate on a separate communication channel 219 using any suitable radio access technology (e.g., Bluetooth, Zigbee, cellular, etc.). The receiver 208 and the transmitter 204 may alternatively communicate via in-band signaling using characteristics of the wireless field 205.

The receiver 208 may be configured to determine whether an amount of power transmitted by the transmitter 204 and received by the receiver 208 is appropriate for charging the battery 236. In certain aspects, the transmitter 204 may be configured to generate a predominantly non-radiative field with a direct field coupling coefficient (k) for providing energy transfer. Receiver 208 may directly couple to the wireless field 205 and may generate an output power for storing or consumption by a battery (or load) 236 coupled to the output or receive circuitry 210.

The receiver 208 may further include a controller 250 configured similarly to the transmit controller 240 as described above for managing one or more aspects of the receiver 208. The receiver 208 may further include a memory (not shown) configured to store data, such as instructions for causing the controller 250 to perform particular functions, such as those related to management of wireless power transfer.

As discussed above, transmitter 204 and receiver 208 may be separated by a distance and may be configured according to a mutual resonant relationship to minimize transmission losses between the transmitter 204 and the receiver 208.

FIG. 3 is a schematic diagram of a portion of the transmit circuitry 206 or the receive circuitry 210 of FIG. 2, in accordance with certain aspects of the present disclosure. As illustrated in FIG. 3, transmit or receive circuitry 350 may include a power transmitting or receiving element 352 and a tuning circuit 360. The power transmitting or receiving element 352 may also be referred to or be configured as an antenna or a “loop” antenna. The term “antenna” generally refers to a component that may wirelessly output or receive energy for coupling to another antenna. The power transmitting or receiving element 352 may also be referred to herein or be configured as a “magnetic” antenna, or an induction coil, a resonator, or a portion of a resonator. The power transmitting or receiving element 352 may also be referred to as a coil or resonator of a type that is configured to wirelessly output or receive power. As used herein, the power transmitting or receiving element 352 is an example of a “power transfer component” of a type that is configured to wirelessly output and/or receive power. The power transmitting or receiving element 352 may include an air core or a physical core such as a ferrite core (not shown in this figure).

When the power transmitting or receiving element 352 is configured as a resonant circuit or resonator with tuning circuit 360, the resonant frequency of the power transmitting or receiving element 352 may be based on the inductance and capacitance. Inductance may be simply the inductance created by a coil and/or other inductor forming the power transmitting or receiving element 352. Capacitance (e.g., a capacitor) may be provided by the tuning circuit 360 to create a resonant structure at a desired resonant frequency. As a non-limiting example, the tuning circuit 360 may comprise a capacitor 354 and a capacitor 356, which may be added to the transmit and/or receive circuitry 350 to create a resonant circuit.

The tuning circuit 360 may include other components to form a resonant circuit with the power transmitting or receiving element 352. As another non-limiting example, the tuning circuit 360 may include a capacitor (not shown) placed in parallel between the two terminals of the circuitry 350. Still other designs are possible. In some aspects, the tuning circuit in the front-end circuit 226 may have the same design (e.g., 360) as the tuning circuit in front-end circuit 232. In other aspects, the front-end circuit 226 may use a tuning circuit design different from the front-end circuit 232.

For power transmitting elements, the signal 358, with a frequency that substantially corresponds to the resonant frequency of the power transmitting or receiving element 352, may be an input to the power transmitting or receiving element 352. For power receiving elements, the signal 358, with a frequency that substantially corresponds to the resonant frequency of the power transmitting or receiving element 352, may be an output from the power transmitting or receiving element 352. Although aspects disclosed herein may be generally directed to resonant wireless power transfer, persons of ordinary skill in the art will appreciate that aspects disclosed herein may be used in non-resonant implementations for wireless power transfer.

In some aspects, when power is wirelessly received by a device (e.g., a medical implant) with a wireless power receiver (e.g., receiver 208) from a wireless power transmitter (e.g., transmitter 204), there may be a method of power control to ensure that the correct amount of power is transferred from the transmitter 204 to the receiver 208. For example, the device with the receiver 208 may be configured to operate or charge at a particular voltage (e.g., 4.2 V). However, generating a fixed strength wireless field 205 by the transmitter 204 may not produce the desired voltage at the receiver 208. For example, the amount of power transferred between the transmitter 204 and the receiver 208 at any given strength of the wireless field 205 may differ based on the distance between (and/or other factors such as materials between, etc.) the transmitter 204 and the receiver 208. Accordingly, the power generated by the receiver 208 for the device may be variable based on one or more factors for the same strength of wireless field 205 from the transmitter 204. For example, a medical implant may be implanted in a person at various distances/positions under the skin and with varying tissue types and thicknesses.

In some aspects, a closed-loop power control scheme may be employed to adjust the strength of the wireless field 205 to ensure that the power (e.g., voltage) at the device being wirelessly powered is the desired power (e.g., desired voltage). For example, in some aspects, the wireless receiver 208 may be configured to actively determine a power level of the power received at the receiver 208, such as, a voltage at the rectifier circuit 234. For example, the controller 250 may be configured to monitor the voltage at the rectifier circuit 234. Depending on whether the voltage at the rectifier circuit 234 is above or below a range of the desired voltage level, the wireless receiver 208 (e.g., as controlled by the controller 250) may transmit feedback information (e.g., as a control signal) (e.g., via communication channel 219 or in-band signaling using the wireless field 205) to the wireless transmitter 204 indicating whether a strength of the wireless field 205 should be increased or decreased. No control signal may be sent if the voltage at the rectifier circuit 234 is within the range of the desired voltage level. The wireless transmitter 204 may receive the control signal and adjust the strength of the wireless field 205 (e.g., by control from the controller 240), accordingly.

Example Safe Wireless Charging with Slow Power Ramp-Up

Biomedical implants are becoming more commonly used for treatment of disease and medical conditions in people and other animals. Examples of biomedical implants include pacemakers, neuromodulation devices, insulin pumps, and the like. Such implants are inserted into the body to release metered doses of medication, to stimulate nerves, and to monitor specific biochemical conditions, for example. Biomedical implants are often battery powered and are expected to operate for a long time (e.g., years to lifetimes) without battery replacement.

Many implants are powered by wireless chargers to avoid the issue of changing batteries and the risk associated with such surgeries. Wireless chargers can work well and can greatly increase the life of the implant. In some cases, however, wireless charging can be difficult, especially when a battery has fully discharged (referred to as a “dead battery”) and the implant is “inert.” In such cases, the implant cannot communicate with the outside world, which can present a problem.

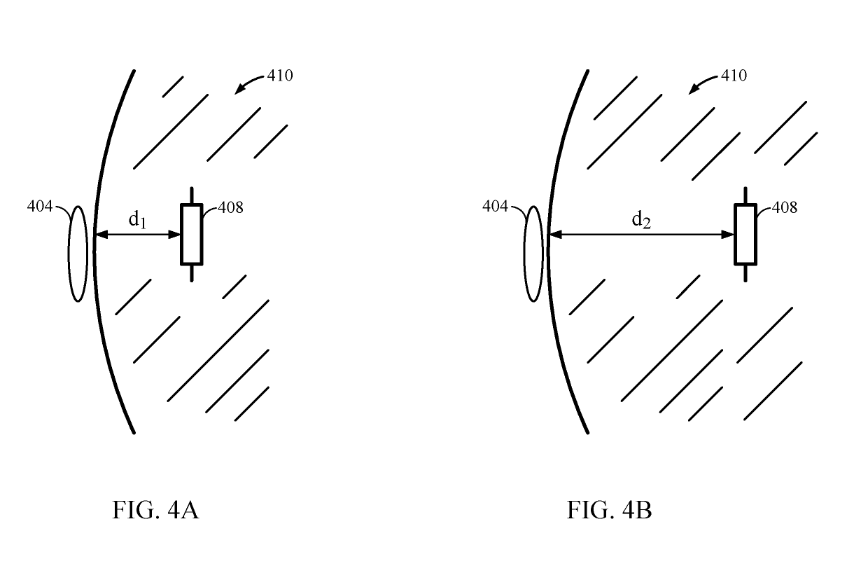

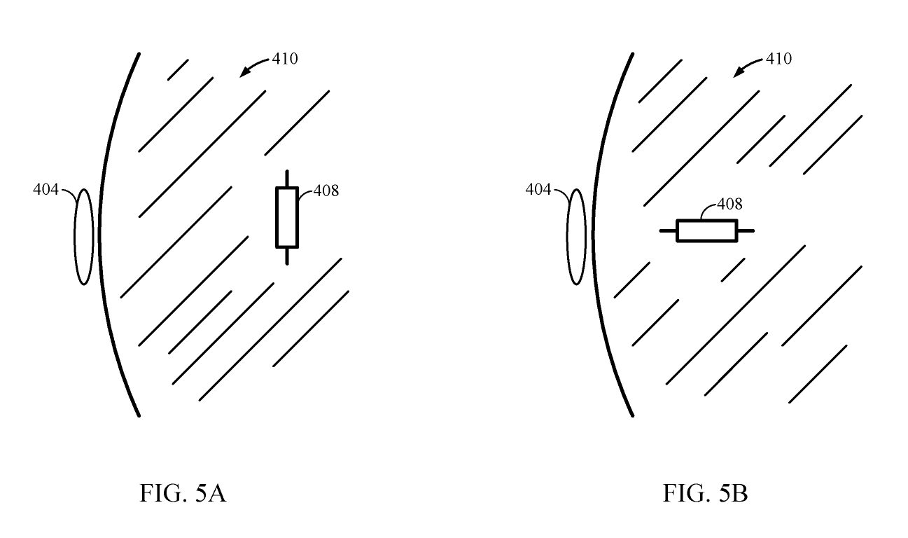

Moreover, implants can change position within a body, which may alter the depth and/or orientation of the implant. For example, FIG. 4A illustrates a wireless charger 404 for wirelessly charging an implantable device 408 (e.g., a biomedical implant) disposed within a patient's body 410 at a distance d₁ from a surface of the body 410. FIG. 4B illustrates the same implantable device 408 located at a distance d₂ from a surface of the patient's body 410, where d₂>d₁. Since implants can vary greatly in position within the body and because animal tissue is an inherently lossy medium for wireless power to propagate through, a wireless charging field that is adequate to charge an implantable device 408 that is 12 cm deep (e.g., d₂ in FIG. 4B) may overload (and even damage) an implantable device 408 at a depth of 1 cm (e.g., d₁ in FIG. 4A). In addition, the orientation of the implant can change, which can alter the implant antenna gain pattern within the body. For example, FIGS. 5A and 5B illustrate the implantable device 408 in two different orientations within the patient's body 410.

All of this leads to an unknown level of the electromagnetic coupling between the wireless charger 404 and the implantable device 408. Communication can alleviate this by allowing closed-loop operation between the wireless charger 404 and the implantable device 408. However, when the battery is dead, communications often cannot come up rapidly; the implantable device 408 may in some cases need to first receive wireless power, then boot, then begin to communicate.

In order to charge a medical implant, the transmitted power received by the implant should be above the minimum charging threshold (p₁). To avoid damaging the implant, the transmitted power received by the implant should be below the maximum charging threshold (p₂). The range p₁ to p₂ may differ per type of implant.

To achieve a received power between p₁ and p₂, the associated wireless charger should transmit a signal power level (P) that is greater than p₁ by the amount of loss (L) that occurs in the body mass between the charger and the implant, including any losses in the antennas. Additionally, P should be no greater than the loss L plus p₂ in order to prevent damage to the implant. These constraints lead to the following inequalities:

p₁≤P−L<p₂

The total loss is an unknown that depends on antenna orientation, body loss, and other factors.

As the implant moves within the body (e.g., moving as the patient bends, as the organs shift during digestion, as an ovary floats within the abdominal cavity, or as gravity affects the abdomen throughout the day), the distance (d) between the implant and the surface of the body can change, as shown in FIGS. 4A and 4B. As d increases, the body mass between the charger and the implant increases. Thus, the loss L increases, since L is a function of d. Likewise, L decreases as d decreases. As L varies, P should be varied to keep the power received at the implant at a level between p₁ and p₂, thus charging without damaging the implant.

When an implant is re-charged before fully draining its battery, the charger may use a signal from the implant to determine the total path loss between the charger and the implant. From this, the charger may set P accordingly. However, if the battery is fully discharged, the implant is “inert” and thus cannot send the signal to the charger.

Aspects of the present disclosure provide techniques and apparatus for wirelessly charging an implant or other wireless power receiver with a dead battery. Certain aspects of the present disclosure ramp up power slowly, in stages. Each stage may be held long enough to allow booting of the implant and thus the opportunity to establish communication. The intensity of the charging field in each stage may be chosen so that no step is so large that the intensity changes from an intensity insufficient to boot the device to an intensity sufficient to damage it.

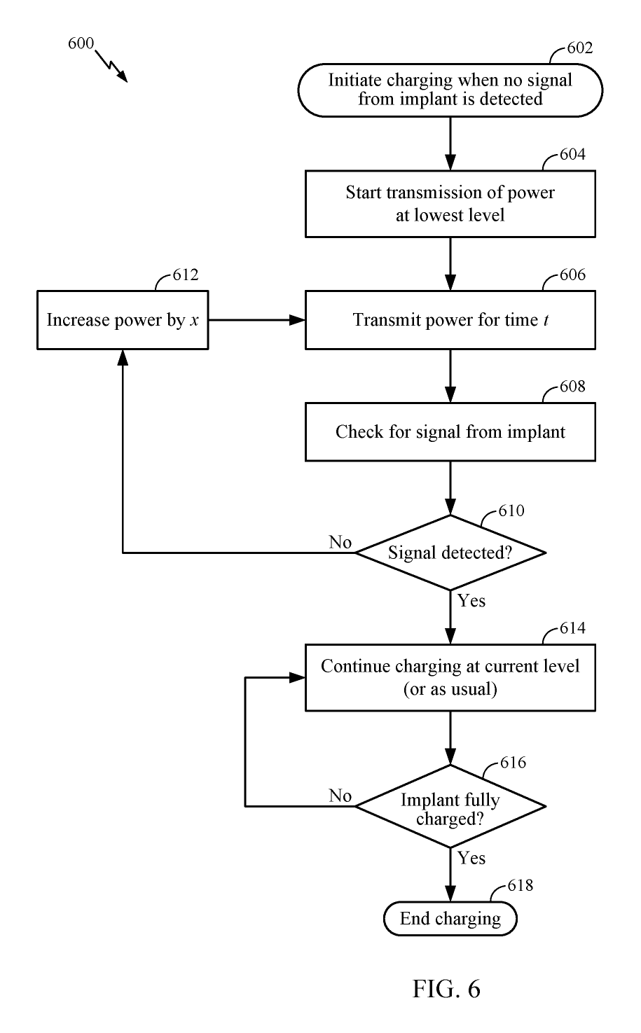

FIG. 6 is a flowchart of example operations 600 for safely wirelessly charging an implant using the step function, illustrating how the power P is ramped up slowly until the charger detects a signal from the implant, in accordance with certain aspects of the present disclosure. The goal of this step function is to charge the implant enough to power the implant back on, allow the implant time to boot, and start receiving the implant's signal (from which charging P is generally determined).

The operations 600 may begin, at block 602, with a determination by the charger that no signal from the implant is detected and a decision to initiate charging. At block 604, the charger may start transmitting power (e.g., a wireless charging field) at an initial value of P. For certain aspects, this initial value of P may be the lowest level that would charge the implant at the closest possible d where the implant may be currently located (e.g., directly under the surface or on the anterior surface of the spine):

P=p₁+Lₘᵢₙ

where Lₘᵢₙ is the minimum possible amount of loss. For other aspects, the charger may determine a statistically significant nominal operating point for the implant and cause this operating point to be stored, either in the charger or external to the charger (e.g., on a network server). In this case, the charger may use this operating point as the initial value of P at block 604.

At block 606, the charger may maintain the selected level for a time t equal to (or greater than) the time it takes to charge a fully discharged battery enough for the battery to power up the implant, so that the implant can start sending a communication signal to the charger. For certain aspects, the time t may be a constant value, while for other aspects, the time t may be a function, which may be based on the contemporary power level output by the charger. At block 608, the charger can check for the signal from the implant. For certain aspects, this signal may be a short packet with minimal communication information (e.g., a beacon or ping, with no data or source/destination address) to save power at the implant. The check at block 608 may be performed at time t, intermittently up until time t, continuously during time t, or after the end of time t. If this value of P is sufficient such that the power received by the implant is above p₁, the implant should now power on, and the charger should detect a signal from the implant and make a decision at block 610. If no signal is detected after time t, the implant may be located deeper in the body (or in some cases, the implant may no longer be functioning for another reason that cannot be addressed by charging).

Increased d means increased L. Thus, P can be increased without the fear of moving above p₂ (which may damage the implant). If no signal is detected at decision block 610, the charger may raise P at block 612 by an increment x in an attempt to go above p₁ of an implant slightly deeper in the body:

P=p₁+Lₓ

where Lₓ is the loss corresponding to the increment x. The increment x may be any suitable function, such as a constant value, a doubled value every iteration, an exponential value, etc. For certain aspects, the charger includes a coil (e.g., power transmitting or receiving element 352) for generating the wireless charging field. The charger adjusts the coil current to control the strength of the charging field. In this case, raising P involves increasing a current flowing in the charger's coil. In each iteration, this may entail, for example, linearly increasing the coil current by a constant value or doubling the coil current.

The new P incremented at block 612 is then transmitted from the charger for time t at block 606, and the signal is checked for again at block 608, as described above. This process is repeated until the signal is detected (or a maximum value of P is reached and still no signal is detected). If the signal is detected at decision block 610, then at block 614, the implant can continue charging at the contemporary power level or can charge as usual (e.g., using closed-loop charging with feedback from the implant). If the implant is not fully charged at decision block 616, the charging may continue at block 614 until the implant is fully charged. After the implant is fully charged at decision block 616, the charger may stop charging at block 618, and the operations 600 may end.

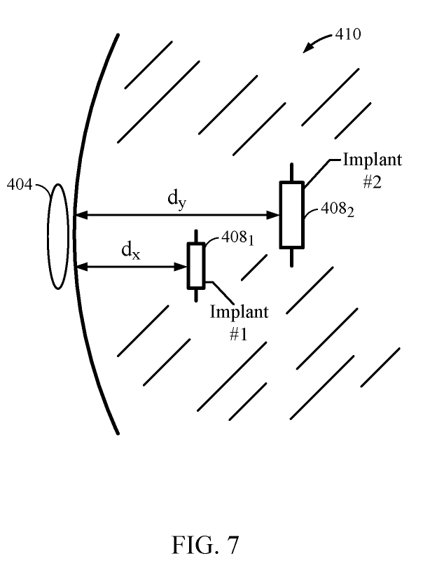

Some patients may have more than one implant in proximity (e.g., an implant in each ovary). For example, FIG. 7 illustrates a first implantable device 408₁ (Implant #1) located at a distance dₓ from a surface of the patient's body 410 and a second implantable device 408₂ (Implant #2) located at a distance d_{y} from the same surface, where d_{y}>dₓ. In this case of multiple implants, the p₁ levels of one implant should not exceed the p₂ level of another implant in the same charging area. If both implants have the ability to exist in the same area, the charger may be moved so that the charger can achieve the p₁ of each implant without ever exceeding a p₂ value of any implant. Generally, the path loss L and P value for each implant can be calculating using the communication signals the charger receives from each implant. If one or more of the multiple implants has gone inert (e.g., has a dead battery), then the ramp-up function (e.g., as described in the operations 600 for FIG. 6) may be performed until the path loss to the inert implant(s) can be determined by powering the implant(s) to a level where the implant(s) can be powered back on and send a communication signal.

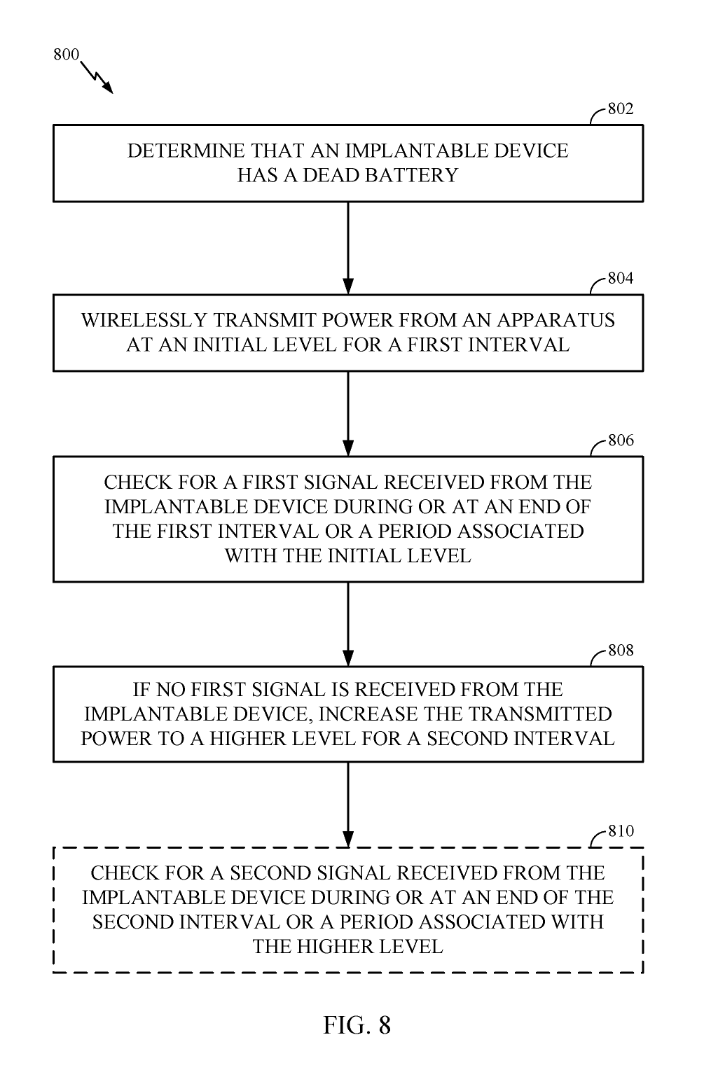

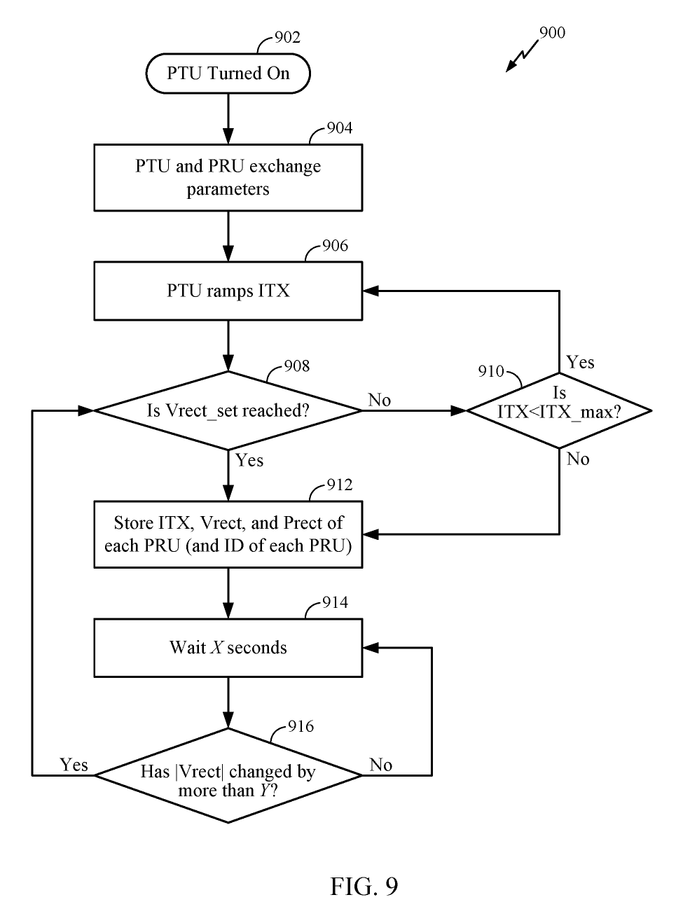

FIG. 8 is a flow diagram of example operations 800 for safely wirelessly charging an implantable device, in accordance with certain aspects of the present disclosure. The operations 800 may be performed by an apparatus, such as a wireless power transmitter (e.g., a PTU).

The operations 800 may begin, at block 802, with the apparatus determining that the implantable device has a dead battery. Based on the determination, the apparatus may wirelessly transmit power at an initial level for a first interval at block 804. At block 806, the apparatus may check for a first signal received from the implantable device during (or at an end of) the first interval or a period associated with the initial level. If no first signal is received from the implantable device, then at block 808, the apparatus may increase the transmitted power to a higher level for a second interval.

According to certain aspects, the operations 800 may further involve the apparatus checking for a second signal received from the implantable device during (or at an end of) the second interval or a period associated with the higher level, at optional block 810. If no second signal is received from the implantable device, the apparatus may repeat the increasing of the transmitted power at block 808 and the checking for the second signal at optional block 810 until: (1) the second signal is received from the implantable device; or (2) the apparatus has wirelessly transmitted the power at a maximum level and no signal was received from the implantable device.

According to certain aspects, the apparatus includes a coil for wirelessly transmitting the power. In this case, increasing the transmitted power to the higher level at block 808 may entail increasing a current in the coil. For certain aspects, the increasing at block 808 involves doubling the current in the coil to obtain the higher level, while for other aspects, this increasing is accomplished by linearly increasing the current in the coil to obtain the higher level.

According to certain aspects, if the first signal or the second signal is received from the implantable device, the operations 800 may further include the apparatus communicating with the implantable device to control wireless charging of the implantable device.

According to certain aspects, the operations 800 may further involve the apparatus: (1) continuing to charge the implantable device at the initial level or wirelessly charging the implantable device using closed-loop control between the apparatus and the implantable device, if the first signal is received from the implantable device; or (2) continuing to charge the implantable device at the higher level or wirelessly charging the implantable device using closed-loop control between the apparatus and the implantable device, if no first signal is received and if the second signal is received from the implantable device.

According to certain aspects, the initial level is a minimum level for the apparatus that could possibly wirelessly charge the implantable device. For certain aspects, a distance between the apparatus and the implantable device is unknown. In this case, the minimum level may be based on: (1) a minimum possible distance between the apparatus and the implantable device; and (2) a power loss associated with the minimum possible distance.

According to certain aspects, the initial level is a default level for the apparatus based on stored data from wirelessly charging the implantable device with the apparatus during previous charging operations (described in more detail below).

According to certain aspects, the first interval is greater than a time to boot the implantable device and to receive the first signal.

Although the present disclosure focuses on implantable devices (e.g., biomedical implants), aspects of the present disclosure may be applied to any wireless power receiver, implantable or otherwise.

### Example Wireless Charging Based on Stored Data

For certain aspects, a PTU (e.g., a wireless power transmitter, such as a charger) may determine a statistically significant nominal operating point for a PRU (e.g., a wireless power receiver, such as an implant) while the PRU's battery is charging well and cause this operating point to be stored, either in the PTU or external to the PTU. Subsequently, during a dead battery scenario, the PTU may default to this stored operating point to charge the device, or this operating point may be the starting point for the initial power level before progressively ramping up the power.

The PTU can either store data associated with wireless charging locally or upload the data to some other location (e.g., computer, cloud, phone, etc.), as described above. Presumably, the user only has to wear a charger for an hour or two at a time to reach full charge. Therefore, sampling the data every few minutes may be appropriate. The sampling rate can be adjusted as desired. For example, if a battery-less implant is used, the sampling frequency may most likely be higher because the patient will be using wireless power in shorter, but more frequent intervals.

Assuming the PTU has power data available, the next step is to determine the nominal operating point. This may be accomplished using any of various suitable statistical techniques, such as averaging all the measured points, selecting the most frequent operating point, selecting the most common operating point when Prect is low (trickle charging a dead battery), select the most common ITX operating point for the PRU that is in dead battery, and the like. The PTU may then set the ITX to a level corresponding to the selected operating point and charge with this value until the PRU began communicating.

FIG. 9 is a flowchart of example operations 900 for wirelessly charging a wireless power receiver according to stored data, in accordance with certain aspects of the present disclosure. The operations 900 may begin, at block 902, when the PTU is turned on. At block 904, the PTU and the PRU may exchange parameters. These parameters may include, for example, the desired DC voltage at the PRU (Vrect_set). At block 906, the PTU may ramp up the ITX.

At decision block 908, if Vrect_set has not yet been reached, then the PTU may determine at decision block 910 whether the ITX is less than the maximum transmit coil current (ITX_max). If true, the PTU may continue ramping up the ITX at block 906. This process is repeated until Vrect_set has been reached or the ITX is greater than or equal to the ITX_max. In either case, the PTU causes data to be stored at block 912, either at the PTU or external to the PTU (e.g., computer, cloud, phone, etc.). The data may include an identifier (ID) of each PRU, the actual DC voltage (Vrect) of each PRU, the actual power (Prect) of each PRU, and/or the ITX of the PTU. At block 914, the PTU may delay for a predetermined amount of time (e.g., by waiting X seconds). At decision block 916, the PTU may determine whether the absolute value of Vrect has changed by more than a particular voltage Y. If not, then the PTU may return to block 914 and wait again, until the value at decision block 916 is true. After the absolute value of Vrect has changed by more than Y, the PTU may return to decision block 908 and determine whether Vrect_set has been reached.

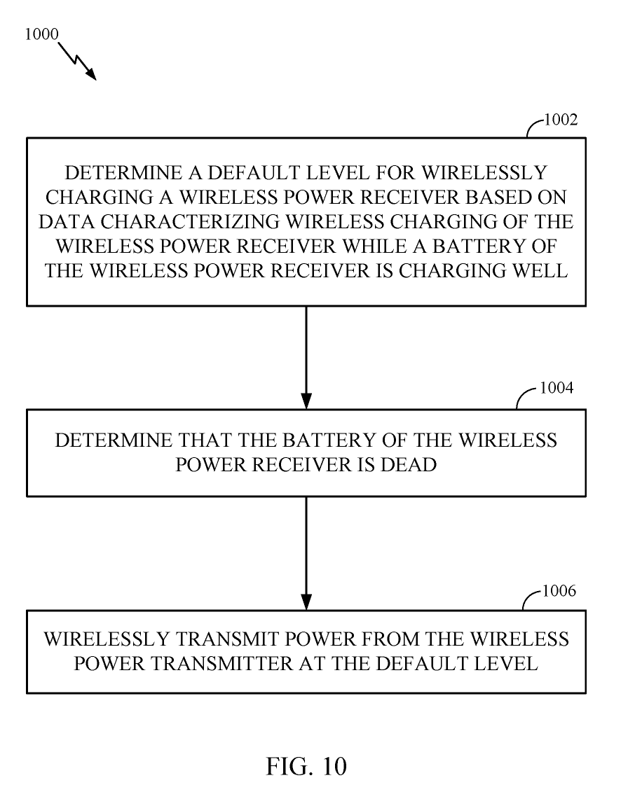

FIG. 10 is a flow diagram of example operations 1000 for wirelessly charging a wireless power receiver, in accordance with certain aspects of the present disclosure. The operations 1000 may be performed, for example, by a wireless power transmitter (e.g., a PTU).

The operations 1000 may begin, at block 1002, with the wireless power transmitter determining a default level for wirelessly charging the wireless power receiver based on data characterizing wireless charging of the wireless power receiver while a battery of the wireless power receiver is charging well (e.g., a good battery). At block 1004, the wireless power transmitter may determine that the battery of the wireless power receiver is dead. Based on the determination of the dead battery at block 1004, the wireless power transmitter may wirelessly transmit power at the default level at block 1006.

According to certain aspects, the operations 1000 may further include the wireless power transmitter receiving parameters from the wireless power receiver, wherein the data includes the received parameters. For certain aspects, the received parameters include at least one of a voltage setpoint, a voltage value, or a power value for the wireless power receiver.

According to certain aspects, the operations 1000 may further entail storing the data at the wireless power transmitter. For other aspects, the operations 1000 may further involve uploading the data from the wireless power transmitter to a remote apparatus (e.g., a network server).

According to certain aspects, the wireless power transmitter includes a coil for wirelessly transmitting the power. In this case, the data may include different values of current in the coil (e.g., at different times). For certain aspects, determining the default level at block 1002 may include averaging the different values of the current in the coil, selecting a most frequent value of the current in the coil, or selecting a most common value of the current in the coil when a power value of the wireless power receiver is relatively low.

According to certain aspects, the wireless power receiver is an implantable medical device.

According to certain aspects, the operations 1000 may further include the wireless power transmitter receiving a signal from the wireless power receiver after wirelessly transmitting power at the default level and, based on reception of the signal, communicating with the wireless power receiver to control wireless charging of the wireless power receiver.

### Example Wireless Charging Based on OVP

For other aspects, a PTU (e.g., a wireless power transmitter, such as a charger) may determine that a PRU (e.g., a wireless power receiver, such as an implant) is likely in a dead battery scenario and slowly increase the transmit coil current (ITX), as described above. Rather than waiting to receive a beacon or other such signal from the PRU, the PTU may wait until an overvoltage protection (OVP) signal is received from the PRU. After receiving the OVP signal, the PTU may decrease the ITX (e.g., a certain amount or percentage) and then charge the PRU at that operating point until the PRU can communicate.

In this manner, the PTU may settle at an operating point just below OVP. Depending on the implementation, the operating point can be fine-tuned. For example, after detecting OVP, the PTU can reduce ITX by a certain percentage, which may generally be a good operating point for the PRU.

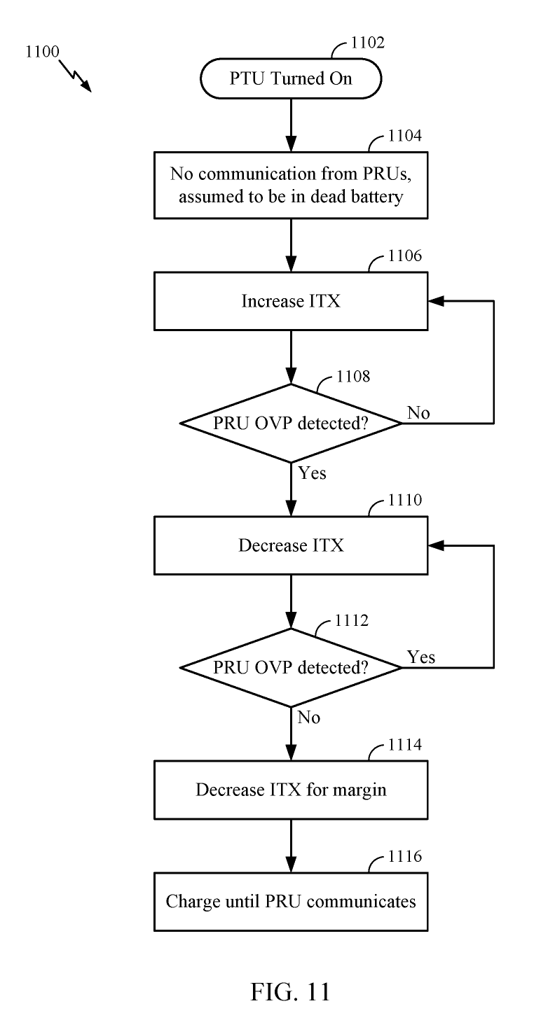

FIG. 11 is a flowchart of example operations 1100 for ramping up power until OVP is detected and then backing off, in accordance with certain aspects of the present disclosure. The operations 1100 may begin, at block 1102, when the PTU is turned on. At block 1104, the PTU determines that that there has been no communication from a PRU and assumes that the PRU is inert with a dead battery. At block 1106, the PTU increases the transmit coil current (ITX) until a PRU OVP condition is detected at block 1108. After the PRU OVP condition occurs at block 1108, the PTU decreases the ITX at block 1110 until the PRU OVP condition is no longer detected at block 1112. In other words, the PTU backs off the ITX from the OVP condition. After the PRU OVP condition is no longer detected at block 1112, the PTU may decrease the ITX still further to have a desired margin at block 1114. This margin may be a particular absolute amount of current or a percentage of the ITX. At block 1116, the PTU may charge at the resulting ITX with the margin until the PRU begins communicating again. At this point, closed-loop charging may resume.

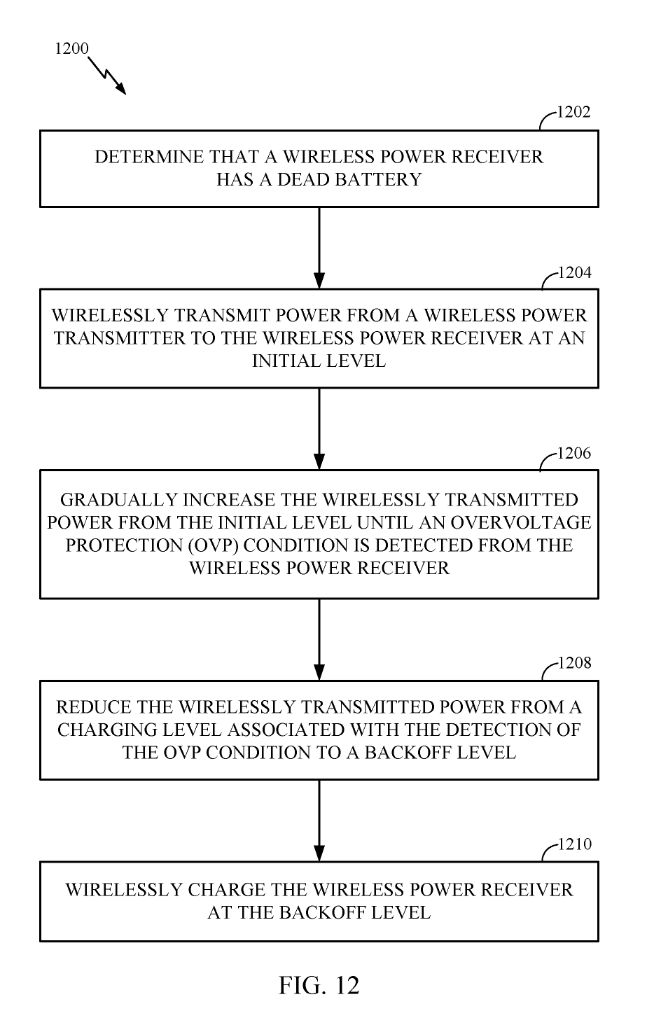

FIG. 12 is a flow diagram of example operations 1200 for wirelessly charging a wireless power receiver, in accordance with certain aspects of the present disclosure. The operations 1200 may be performed, for example, by a wireless power transmitter (e.g., a PTU).

The operations 1200 may begin, at block 1202, with the wireless power transmitter determining that the wireless power receiver has a dead battery. Based on the determination at block 1202, the wireless power transmitter may wirelessly transmit power to the wireless power receiver at an initial level at block 1204. At block 1206, the wireless power transmitter may gradually increase the wirelessly transmitted power from the initial level until an overvoltage protection (OVP) condition is detected from the wireless power receiver. Based on the detection of the OVP condition at block 1206, the wireless power transmitter may reduce the wirelessly transmitted power from a charging level associated with the detection of the OVP condition to a backoff level at block 1208. At block 1210, the wireless power transmitter may wirelessly charge the wireless power receiver at the backoff level.

According to certain aspects, the wireless power transmitter includes a coil for wirelessly transmitting the power. In this case, reducing the wirelessly transmitted power at block 1208 may entail decreasing a current in the coil. For certain aspects, the reducing at block 1208 includes reducing a first value of the current in the coil corresponding to the charging level associated with the detection of the OVP condition by a percentage of the first value to generate a second value of the current in the coil corresponding to the backoff level. For other aspects, the reducing at block 1208 involves reducing a first value of the current in the coil corresponding to the charging level associated with the detection of the OVP condition by a predetermined constant value to generate a second value of the current in the coil corresponding to the backoff level.

According to certain aspects, the operations 1200 further includes the wireless power transmitter receiving a signal from the wireless power receiver after wirelessly charging the wireless power receiver at the backoff level and, based on the reception of the signal, communicating with the wireless power receiver to control wireless charging of the wireless power receiver.

According to certain aspects, the wireless power receiver is an implantable medical device.

The various operations or methods described above may be performed by any suitable means capable of performing the corresponding functions. The means may include various hardware and/or software component(s) and/or module(s), including, but not limited to a circuit, an application-specific integrated circuit (ASIC), or processor. Generally, where there are operations illustrated in figures, those operations may have corresponding counterpart means-plus-function components with similar numbering.

For example, means for determining, means for checking, and/or means for detecting may include a processing system, which may include one or more processors or microcontrollers, such as the controller 240 in FIG. 2. Means for wirelessly transmitting power may include a transmitter (e.g., transmitter 104 in FIG. 1 or transmitter 204 in FIG. 2), which may also be referred to as a power transmitting unit (PTU).

As used herein, the term “determining” encompasses a wide variety of actions. For example, “determining” may include calculating, computing, processing, deriving, investigating, looking up (e.g., looking up in a table, a database, or another data structure), ascertaining, and the like. Also, “determining” may include receiving (e.g., receiving information), accessing (e.g., accessing data in a memory), and the like. Also, “determining” may include resolving, selecting, choosing, establishing, and the like.

As used herein, a phrase referring to “at least one of” a list of items refers to any combination of those items, including single members. As an example, “at least one of: a, b, or c” is intended to cover: a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The various illustrative logical blocks, modules, and circuits described in connection with the present disclosure may be implemented or performed with a general purpose processor, a digital signal processor (DSP), an ASIC, a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

The functions described may be implemented in hardware, software, firmware, or any combination thereof. If implemented in hardware, an example hardware configuration may comprise a processing system in a wireless node. The processing system may be implemented with a bus architecture. The bus may include any number of interconnecting buses and bridges depending on the specific application of the processing system and the overall design constraints. The bus may link together various circuits including a processor, machine-readable media, and a bus interface. The bus interface may be used to connect a network adapter, among other things, to the processing system via the bus. The network adapter may be used to implement the signal processing functions of the physical (PHY) layer. In the case of a user terminal, a user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further.

The processing system may be configured as a general-purpose processing system with one or more microprocessors providing the processor functionality and external memory providing at least a portion of the machine-readable media, all linked together with other supporting circuitry through an external bus architecture. Alternatively, the processing system may be implemented with an ASIC with the processor, the bus interface, the user interface in the case of an access terminal), supporting circuitry, and at least a portion of the machine-readable media integrated into a single chip, or with one or more FPGAs, PLDs, controllers, state machines, gated logic, discrete hardware components, or any other suitable circuitry, or any combination of circuits that can perform the various functionality described throughout this disclosure. Those skilled in the art will recognize how best to implement the described functionality for the processing system depending on the particular application and the overall design constraints imposed on the overall system.

It is to be understood that the claims are not limited to the precise configuration and components illustrated above. Various modifications, changes and variations may be made in the arrangement, operation and details of the methods and apparatus described above without departing from the scope of the claims.

What is claimed is:

1. A method for safely wirelessly charging an implantable device, with an apparatus, the method comprising:
determining that the implantable device has a dead battery;
based on the determination, wirelessly transmitting power from the apparatus at an initial level for a first interval;
checking for a first signal received from the implantable device during or at an end of the first interval or a period associated with the initial level; and
if no first signal is received from the implantable device, increasing the transmitted power to a higher level for a second interval.

2. The method of claim 1, further comprising:
checking for a second signal received from the implantable device during or at an end of the second interval or a period associated with the higher level; and
if no second signal is received from the implantable device, repeating the increasing of the transmitted power and the checking for the second signal until:
the second signal is received from the implantable device; or
the apparatus has wirelessly transmitted the power at a maximum level and no signal was received from the implantable device.

3. The method of claim 2, further comprising:
communicating with the implantable device to control wireless charging of the implantable device, if the first signal or the second signal is received from the implantable device.

4. The method of claim 2, further comprising:
if the first signal is received from the implantable device, continuing to charge the implantable device at the initial level or wirelessly charging the implantable device using closed-loop control between the apparatus and the implantable device; or
if no first signal is received and if the second signal is received from the implantable device, continuing to charge the implantable device at the higher level or wirelessly charging the implantable device using closed-loop control between the apparatus and the implantable device.

5. The method of claim 1, wherein the apparatus comprises a coil for wirelessly transmitting the power and wherein increasing the transmitted power to the higher level comprises increasing a current in the coil.

6. The method of claim 5, wherein the increasing comprises doubling the current in the coil to obtain the higher level.

7. The method of claim 5, wherein the increasing comprises linearly increasing the current in the coil to obtain the higher level.

8. The method of claim 1, wherein the initial level comprises a minimum level for the apparatus that could wirelessly charge the implantable device.

9. The method of claim 8, wherein a distance between the apparatus and the implantable device is unknown and wherein the minimum level is based on a minimum possible distance between the apparatus and the implantable device and a power loss associated with the minimum possible distance.

10. The method of claim 1, wherein the initial level comprises a default level for the apparatus based on stored data from wirelessly charging the implantable device with the apparatus during previous charging operations.

11. The method of claim 1, wherein the first interval is greater than a time to boot the implantable device and to receive the first signal.

12. An apparatus for safely wirelessly charging an implantable device, the apparatus comprising:
a power transmitting element for wirelessly transmitting power;
transmit circuitry coupled to and configured to drive the power transmitting element; and
a processing system coupled to the transmit circuitry and configured to:
determine that the implantable device has a dead battery;
control the transmit circuitry to wirelessly transmit power from the power transmitting element at an initial level for a first interval, based on the determination of the dead battery;
check for a first signal received from the implantable device during or at an end of the first interval or a period associated with the initial level; and
control the transmit circuitry to increase the wirelessly transmitted power to a higher level for a second interval, if no first signal is received from the implantable device.

13. The apparatus of claim 12, wherein the processing system is further configured to:
check for a second signal received from the implantable device during or at an end of the second interval or a period associated with the higher level; and
repeat the increasing of the wirelessly transmitted power and the checking for the second signal, if no second signal is received from the implantable device, until:
the second signal is received from the implantable device; or
the apparatus has wirelessly transmitted the power at a maximum level and no signal was received from the implantable device.

14. The apparatus of claim 13, wherein the processing system is further configured to communicate with the implantable device to control wireless charging of the implantable device, if the first signal or the second signal is received from the implantable device.

15. The apparatus of claim 13, wherein the processing system is further configured to:
control the transmit circuitry to continue to charge the implantable device at the initial level or wirelessly charge the implantable device using closed-loop control between the apparatus and the implantable device, if the first signal is received from the implantable device; or
control the transmit circuitry to continue to charge the implantable device at the higher level or wirelessly charge the implantable device using closed-loop control between the apparatus and the implantable device, if no first signal is received and if the second signal is received from the implantable device.

16. The apparatus of claim 12, wherein the power transmitting element comprises a coil for wirelessly transmitting the power and wherein the processing system is configured to control increasing the wirelessly transmitted power to the higher level by controlling the transmit circuitry to increase a current in the coil.

17. The apparatus of claim 16, wherein the processing system is configured to control the increasing by controlling the transmit circuitry to double the current in the coil to obtain the higher level.

18. The apparatus of claim 16, wherein the processing system is configured to control the increasing by controlling the transmit circuitry to linearly increase the current in the coil to obtain the higher level.

19. The apparatus of claim 12, wherein the initial level comprises a minimum level for the apparatus that could wirelessly charge the implantable device.

20. The apparatus of claim 19, wherein a distance between the apparatus and the implantable device is unknown and wherein the minimum level is based on a minimum possible distance between the apparatus and the implantable device and a power loss associated with the minimum possible distance.

21. The apparatus of claim 12, wherein the initial level comprises a default level for the apparatus based on stored data from wirelessly charging the implantable device with the apparatus during previous charging operations.

22. The apparatus of claim 12, wherein the first interval is greater than a time to boot the implantable device and to receive the first signal.

23. A wireless power transmitter for wirelessly charging a wireless power receiver, the wireless power transmitter comprising:
a power transmitting element for wirelessly transmitting power;
transmit circuitry coupled to and configured to drive the power transmitting element; and
a processing system coupled to the transmit circuitry and configured to:
determine that the wireless power receiver has a dead battery;
control the transmit circuitry to wirelessly transmit power from the power transmitting element at an initial level, based on the determination of the dead battery;
control the transmit circuitry to gradually increase the wirelessly transmitted power from the initial level until an overvoltage protection (OVP) condition is detected from the wireless power receiver;
control the transmit circuitry to reduce the wirelessly transmitted power from a charging level associated with the detection of the OVP condition to a backoff level, based on the detection of the OVP condition; and
control the transmit circuitry to wirelessly charge the wireless power receiver at the backoff level.

24. The wireless power transmitter of claim 23, wherein the power transmitting element comprises a coil for wirelessly transmitting the power and wherein the processing system is configured to control the transmit circuitry to reduce the wirelessly transmitted power by controlling the transmit circuitry to decrease a current in the coil.

25. The wireless power transmitter of claim 24, wherein the processing system is configured to control the transmit circuitry to decrease the current by controlling the transmit circuit to reduce a first value of the current in the coil corresponding to the charging level associated with the detection of the OVP condition by a percentage of the first value to generate a second value of the current in the coil corresponding to the backoff level.

26. A wireless power transmitter for wirelessly charging a wireless power receiver, the wireless power transmitter comprising:
a power transmitting element for wirelessly transmitting power;
transmit circuitry coupled to and configured to drive the power transmitting element; and
a processing system coupled to the transmit circuitry and configured to:
determine a default level for wirelessly charging the wireless power receiver based on data characterizing wireless charging of the wireless power receiver while a battery of the wireless power receiver is charging well;
determine that the battery of the wireless power receiver is dead; and
control the transmit circuitry to wirelessly transmit power from the power transmitting element at the default level, based on the determination that the battery is dead.

27. The wireless power transmitter of claim 26, further comprising receive circuitry configured to receive parameters from the wireless power receiver, wherein the data includes the received parameters and wherein the received parameters comprise at least one of a voltage setpoint, a voltage value, or a power value for the wireless power receiver.

28. The wireless power transmitter of claim 26, further comprising a memory configured to store the data at the wireless power transmitter.

29. The wireless power transmitter of claim 26, wherein the power transmitting element comprises a coil for wirelessly transmitting the power and wherein the data includes different values of current in the coil.

30. The wireless power transmitter of claim 29, wherein the processing system is configured to determine the default level by:
averaging the different values of the current in the coil;
selecting a most frequent value of the current in the coil; or
selecting a most common value of the current in the coil when a power value of the wireless power receiver is relatively low.
